(19) United States
(12) Patent Application Publication
(10) Pub. No.: US 2025/0366759 A1
(43) Pub. Date: Dec. 4, 2025

(54) IMPLANTABLE PROBE APPARATUS

(71) Applicant: NEUROXESS TECHNOLOGY (SHANGHAI) CO., LTD., SHANGHAI (CN)

(72) Inventors: Lei PENG, Shanghai (CN); Zheng TAN, Shanghai (CN)

(73) Assignee: NEUROXESS TECHNOLOGY (SHANGHAI) CO., LTD., SHANGHAI (CN)

(21) Appl. No.: 19/108,103

(22) PCT Filed: Aug. 23, 2023
(86) PCT No.: PCT/CN2023/114450

§ 371 Date: Feb. 28, 2025

(30) Foreign Application Priority Data
Aug. 31, 2022 (CN) 202211054781.3

(51) Int. Cl. A61B 5/293; A61B 5/263; G06F 3/01

(52) U.S. Cl. CPC A61B 5/293; A61B 5/263; A61B 2562/046; A61B 2562/125; A61B 2562/164; G06F 3/015

(57) ABSTRACT

An implantable probe apparatus includes: a flexible substrate, which includes a first part and a plurality of second parts separated from each other; a probe pad array, which includes a plurality of contact pads that are formed in the first part; a plurality of electrodes, which are formed in respective tail end sections of the plurality of second parts away from the first part; and a plurality of leads, which are formed in the plurality of second parts to electrically connect the plurality of electrodes to the corresponding contact pads respectively; where each second part in the plurality of second parts includes N stages of segments, the N^{th} stage of segments include the respective tail end sections of the plurality of second parts, a plurality of branches are branched from each segment in the n^{th} stage of segments to serve as the (n+1)^{th} stage of segments.

### CROSS REFERENCE TO RELATED APPLICATIONS

The disclosure claims the priority to Chinese Patent Application No. 2022110547813 filed on Aug. 31, 2022, which is incorporated herein by reference in its entirety.

### TECHNICAL FIELD

The disclosure relates to the technical field of microelectronic packaging and interconnection, and in particular to an implantable probe apparatus and a preparation method therefor, an electrode apparatus, and an electronic device.

### BACKGROUND ART

Brain-computer interfaces, which are sometimes referred to as “brain ports” or “brain-computer fusion perception”, are direct connection paths established between the human or animal brains (or cultures of brain cells) and external devices. As a multidisciplinary technology, the brain-computer interfaces have attracted extensive attention from the scientific research and industrial communities throughout the world. Serving as a branch of the brain-computer interface, a flexible probe apparatus is considered to be “the final form of the brain-computer interface” thanks to the superior biocompatibility thereof.

### SUMMARY

According to an aspect, the disclosure provides an implantable probe apparatus, including: a flexible substrate, which includes a first part and a plurality of second parts separated from each other, where the first part is located at a first end of the implantable probe apparatus, and the plurality of second parts extend from the first part to a second end of the implantable probe apparatus, the second end being opposite to the first end; a probe pad array, which includes a plurality of contact pads that are formed in the first part; a plurality of electrodes, which are formed in respective tail end sections of the plurality of second parts away from the first part, the tail end sections serving as probes to be implanted into the brain of an organism; and a plurality of leads, which are formed in the plurality of second parts to electrically connect respective electrodes in the plurality of electrodes to corresponding contact pads in the plurality of contact pads respectively, where each second part in the plurality of second parts includes N stages of segments, the N stages of segments are arranged sequentially in a direction from the first end to the second end, and the N^{th} stage of segments of the plurality of second parts include the tail end sections of the plurality of second parts, where N represents an integer greater than or equal to 2; and where a plurality of branches are branched from each segment in the n^{th} stage of segments to serve as the (n+1)th stage of segments, the leads formed in each segment of the (n+1)th stage of segments are subsets of the leads formed in the n^{th} stage of segments, where n represents an integer and 0<n<N.

According to an aspect, the disclosure provides an electrode apparatus, including an implantable probe apparatus as described in any one of the above aspects; and a data adapter, which is electrically connected to a plurality of contact pads in the probe pad array and configured to transmit signals to the plurality of contact pads or receive signals from the plurality of contact pads.

According to an aspect, the disclosure provides an electronic device, including an electrode apparatus described above.

According to an aspect, the disclosure provides a method for preparing an implantable probe apparatus, the method including: forming a first flexible substrate layer on a support substrate, the first flexible substrate layer including a first region and a plurality of second regions, where the first region is located at a first end of the implantable probe apparatus, and the plurality of second regions extend from the first region to a second end of the implantable probe apparatus, the second end being opposite to the first end; forming a metal pattern layer on the first flexible substrate layer, the metal pattern layer including a probe pad array, a plurality of electrodes and a plurality of leads, where the probe pad array includes a plurality of contact pads, the plurality of contact pads are formed in the first region, the plurality of electrodes are formed in respective tail end sections of the plurality of second regions away from the first region, and the plurality of leads are formed in the plurality of second regions to electrically connect the corresponding electrodes in the plurality of electrodes to the respective contact pads in the plurality of contact pads respectively; covering the first flexible substrate layer formed with the metal pattern layer by a second flexible substrate layer; etching the second flexible substrate layer and the first flexible substrate layer to expose the plurality of contact pads and the plurality of electrodes, and forming a first part corresponding to a pattern of the first region and a plurality of second parts corresponding to patterns of the plurality of second regions, where the plurality of second parts are separated from each other, each second part includes N stages of segments, the N stages of segments are arranged sequentially in a direction from the first end to the second end, the N^{th} stage of segments of the plurality of second parts include tail end sections corresponding to the tail end sections of the plurality of second regions, and the tail end sections of the plurality of second parts function as probes for implantation into the brain of an organism, where N represents an integer greater than or equal to 2; and where a plurality of branches are branched from each segment in the n^{th} stage of segments to serve as the (n+1) th stage of segments, and the leads formed in each segment of the (n+1) th stage of segments are subsets of the leads formed in the n^{th} stage of segments, where n represents an integer and 0<n<N; and removing a part of the support substrate except for a first support substrate part, the first support substrate part corresponding to the first part.

These and other aspects of the disclosure will be clear from the embodiments described below, and will be clarified with reference to the embodiments described below.

### BRIEF DESCRIPTION OF THE DRAWINGS

More details, features and advantages of the disclosure are disclosed in the following description of exemplary embodiments with reference to the accompanying drawings, in which:

FIG. 1 is a schematic structural diagram of a probe apparatus provided in the related art;

FIG. 2 is a schematic structural diagram of an implantable probe apparatus according to some embodiments of the disclosure;

FIG. 3 is a schematic structural diagram of a tail end section of a second part in an implantable probe apparatus according to some embodiments of the disclosure;

FIG. 4 is a schematic structural diagram of a partial cross section of an implantable probe apparatus according to some embodiments of the disclosure in an extension direction from a first end to a second end;

FIG. 5 is an exploded schematic structural diagram of an electrode apparatus according to some embodiments of the disclosure;

FIG. 6 is a schematic flowchart of a method for preparing an implantable probe apparatus according to some embodiments of the disclosure; and

FIG. 7 is a schematic diagram of a process for preparing an implantable probe apparatus according to some embodiments of the disclosure.

### DETAILED DESCRIPTION OF EMBODIMENTS

Only some exemplary embodiments will be briefly described below. As can be appreciated by those skilled in the art, the described embodiments can be modified in various ways without departing from the spirit or scope of the disclosure. Accordingly, the accompanying drawings and the description are considered illustrative in nature rather than limited.

In the related art, a brain electrode is implanted into the brain of an organism by using a probe apparatus. The flexible probe apparatus includes contact pads and a plurality of probes extending from the contact pads, and a tail end of each probe is designed to be flexible for implantation into the brain of the organism. The probes are arranged at intervals in a one-dimensional manner, distances between the probes are fixed, and thus a range of a coverable brain region is relatively limited. If it is required to cover a larger brain region, a plurality of probe apparatuses are often needed, and thus rear-end interfaces of the plurality of probe apparatuses are left on the head.

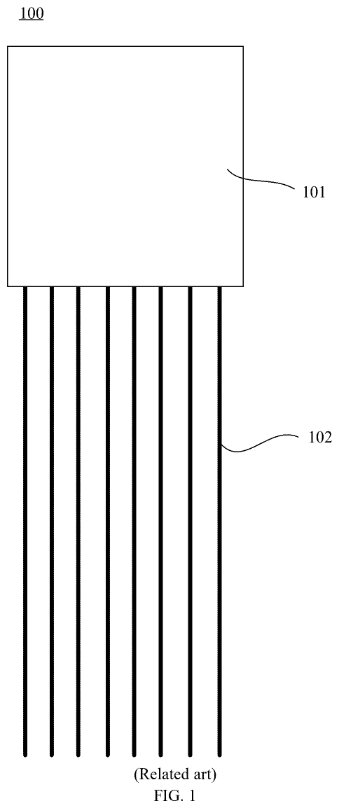

FIG. 1 is a schematic structural diagram of a probe apparatus 100 provided in the related art. As shown in FIG. 1, the probe apparatus 100 includes a probe pad array 101 and a plurality of probes 102. A front end of each probe 102 is connected to the probe pad array 101, and a tail end thereof is designed to be flexible for implantation into the brain of an organism. The probes 102 are arranged at intervals in a one-dimensional manner, and distances between the probes 102 are fixed, such that the probes 102 are linearly and fixedly distributed in a one-dimensional manner during the implantation in most cases, and accordingly implantation locations of the probes 102 cannot be selected according to actual requirements. In addition, a range of brain regions coverable by conventional probe apparatuses 100 is relatively limited, and if it is required to cover a larger brain region, a plurality of probe apparatuses 100 are needed in most cases, and thus rear-end interfaces of the plurality of probe apparatuses 100 are left on the head, which causes large skull injuries and is not conducive to clinical use.

In view of this, the disclosure provides an implantable probe apparatus and a preparation method therefor, an electrode apparatus and an electronic device, in order to increase the area of the brain region coverable by a single probe apparatus, decrease the number of the rear-end interfaces connected to the probe apparatuses, and reduce the skull injuries to a recipient.

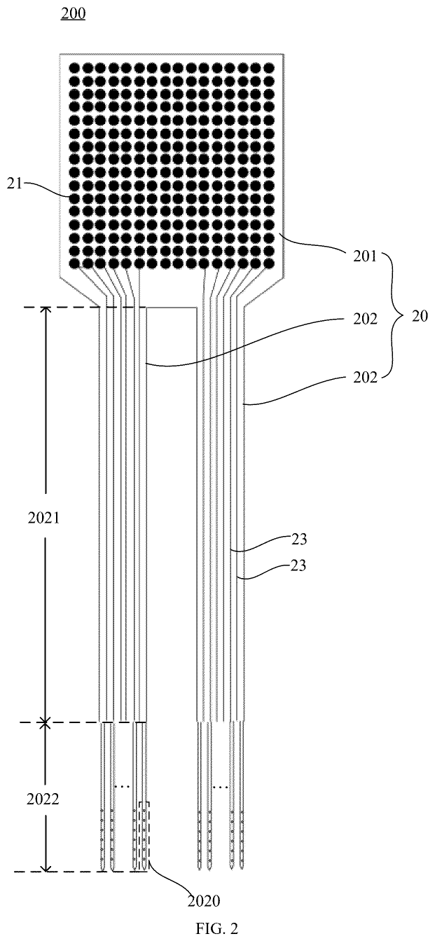

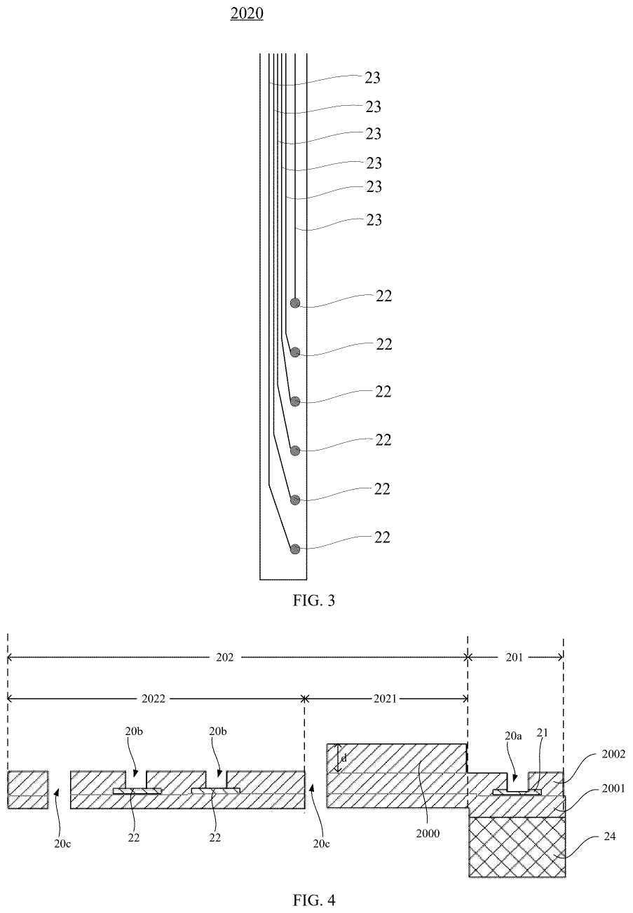

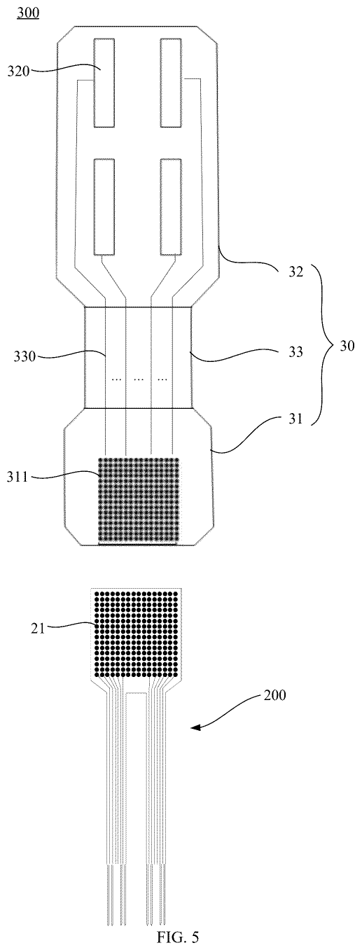

Reference is made to FIGS. 2, 3 and 4. FIG. 2 is a schematic structural diagram of an implantable probe apparatus 200 according to some embodiments of the disclosure. FIG. 3 is a schematic structural diagram of a tail end section 2020 of a second part in an implantable probe apparatus according to some embodiments of the disclosure. FIG. 4 is a schematic structural diagram of a partial cross section of an implantable probe apparatus 200 according to some embodiments of the disclosure in an extension direction from a first end to a second end.

It should be noted that FIGS. 2, 3 and 4 are merely used for schematically showing the features of some structures and do not limit the actual numbers and sizes of these structures. For example, only two second parts, two stages of segments of each second part, and leads and other structures therein are shown schematically in FIG. 2, where the number of the second parts, the number of various stages of segments of each second part and the number of the leads do not represent the number of these structures in an actual product. Similarly, the number of electrodes and the number of the leads in FIG. 3 also do not represent the number of these structures in the actual product and are not intended to limit the disclosure. In FIG. 4, only a cross section involving two electrodes and one contact pad is taken schematically, and a cross section involving the leads is not shown (the leads are located in other cross sections).

In an aspect, the disclosure provides an implantable probe apparatus. As shown in FIGS. 2 and 3, the implantable probe apparatus 200 includes a flexible substrate 20, and a probe pad array, a plurality of electrodes 22 and a plurality of leads 23 that are located in the flexible substrate 20.

The flexible substrate 20 includes a first part 201 and a plurality of second parts 202 separated from each other. The first part 201 is located at a first end of the implantable probe apparatus 200, and the plurality of second parts 202 extend from the first part 201 to a second end of the implantable probe apparatus 200, the second end being opposite to the first end.

The flexible substrate 20 is configured to carry and protect the probe pad array, the plurality of electrodes 22 and the plurality of leads 23. In some embodiments, as shown in FIG. 4, the flexible substrate 20 may include a first flexible substrate layer 2001 and a second flexible substrate layer 2002 that are arranged in a stacked manner, and the probe pad array, the plurality of electrodes 22 and the plurality of leads 23 are located between the first flexible substrate layer and the second flexible substrate layer. In some examples, the first flexible substrate layer 2001 and the second flexible substrate layer 2002 may be made of the same or different materials, and may specifically be made from a polyimide (PI) material.

The probe pad array includes a plurality of contact pads 21, and the plurality of contact pads 21 are formed in the first part 201 of the flexible substrate for electrical connection with an external circuit. In the example of FIG. 4, contact holes 20a for exposing the plurality of contact pads 21 are formed in the second flexible substrate layer 2002 to enable the contact pads 21 to be electrically connected to the external circuit.

The plurality of electrodes 22 are formed in tail end sections 2020 of the plurality of second parts 202 away from the first part 201, and the tail end sections 2020 function as probes to be implanted into the brain of the organism, where the plurality of electrodes 21 are configured to collect brain signals or output stimulation signals to brain tissues. In the example of FIG. 4, the second flexible substrate layer 2002 is provided with connecting holes 20b for exposing the plurality of electrodes 22 to enable the plurality of electrodes 22 to come into contact with the brain tissues in order to collect the brain signals or output the stimulation signals to the brain tissues.

The plurality of leads 23 are formed in the plurality of second parts 202 to electrically connect corresponding electrodes 22 in the plurality of electrodes 22 to respective contact pads 21 in the plurality of contact pads 21 respectively.

The plurality of electrodes 22 are in one-to-one correspondence with the plurality of leads 23, and each electrode 22 is connected to one contact pad 21 by means of one lead 23 corresponding thereto and is thus connected to the external circuit. In some examples, the plurality of contact pads 21 are connected to a chip by means of a data adapter, and thus the plurality of electrodes 22 are electrically connected to a circuit of the chip.

According to some embodiments, each second part 202 in the plurality of second parts 202 of the flexible substrate 20 includes N stages of segments. The N stages of segments are arranged sequentially in a direction from the first end of the implantable probe apparatus 200 to the second end of the implantable probe apparatus 200, and the N^{th} stage of segments of the plurality of second parts 202 include the tail end sections 2020 of the plurality of second parts 202, where N represents an integer greater than or equal to 2. In other words, tail ends of the segments in the last stage of segments of each second part 202 are the tail end sections 2020 of the second part 202. The last stage of segments of each second part 202 may be referred to as the probes, and the tail end sections 2020 thereof may be referred to as implanted probe parts.

In each second part 202 of the flexible substrate 20, a plurality of branches are branched from each segment in the n^{th} stage of segments to serve as the (n+1)^{th} stage of segments. In other words, the plurality of branches that are branched from each segment in the n^{th} stage of segments are a plurality of segments in the (n+1)^{th} stage of segments. Therefore, the number of the segments in the (n+1)^{th} stage of segments is greater than the number of segments in the n^{th} stage of segments, and the leads formed in each segment in the (n+1)^{th} stage of segments are subsets of the leads formed in the n^{th} stage of segments, where n represents an integer and 0<n<N.

In the example of FIG. 2, each second part 202 of the flexible substrate 20 includes two stages of segments, which are the first stage of segment 2021 and the second stage of segments 2022 respectively, that is, N is equal to 2. The first stage of segment 2021 of each second part 202 includes one segment, and a plurality of branches are branched from the one segment in the first stage of segment 2021 to form a plurality of segments of the second stage of segments 2022. The tail end sections 2020 of the segments in the second stage of segments 2022 function as the probes for implantation into the brain of the organism, and each tail end section 2020 is provided with the plurality of electrodes 21 for collecting the brain signals or outputting the stimulation signals to the brain tissues.

As shown in FIGS. 2 and 3, the plurality of leads 23 in the first stage of segment 2021 are dispersed into the segments in the second stage of segments 2022 and ultimately connected to the electrodes 22 at the tail ends of the segments of the second stage of segments 2022. Conversely, the leads 23 in the segments of the second stage of segments 2022 are gathered in the first stage of segment 2021 and ultimately connected to the contact pads 21.

According to the embodiments of the disclosure, the second part of the flexible substrate uses a multi-stage segmentation design, the numbers of segments in the various stages of segments are gradually increased sequentially from the first stage of segment to the N^{th} stage of segments, then the number of segments in the last stage of segments (the N^{th} stage of segments) may be much greater than the number of the segment in the first stage of segment (e.g., amplified exponentially), and the tail end regions of the segments in the last stage of segments are configured as probes. In this way, the implantable probe apparatus is provided with a larger number of probes and thus can cover a larger implantation range, so that the coverage area of a single implantable probe apparatus can be increased. Thus, the number of the implantable probe apparatuses required for detecting electroencephalogram signals can be decreased, the number of rear-end adapter interfaces connected to the implantable probe apparatuses can be decreased, and accordingly the skull injuries to the recipient are reduced.

In addition, in the second part of the flexible substrate, the numbers of segments in the various stages of segments are gradually decreased sequentially from the N^{th} stage of segments to the first stage of segment, so that the grouped management of the probes can be facilitated, and the entanglement between the plurality of leads is prevented. For example, the brain of the organism generally includes brain regions such as the hippocampus, the medial temporal lobe and the like in the brain, the probes formed by the tail end sections of each second part of the flexible substrate serve as a large group, and the probes in each large group are configured for implantation into a corresponding brain region of the brain. In this way, the entanglement between the probes of the second parts may be avoided, and the classified management of the collected electroencephalogram signals can be facilitated. By analogy, each brain region may also be graded, stage by stage, into N stages of regions to correspond to the N stages of segments of the second part, and the probes corresponding to the various stages of segments may be implanted into the corresponding stages of regions in the brain region. For example, the probes corresponding to the segment of the first stage of segment are implanted into the respective regions of a first stage of regions in the brain region, the probes corresponding to the segments in the second stage of segments are implanted into the respective regions of a second stage of regions in the brain region, and by analogy, the hierarchical management of the probes and the detected signals thereby can be achieved.

As shown in FIG. 4, according to some embodiments, the plurality of second parts 202 of the flexible substrate 20 include a plurality of through holes 20c running through the flexible substrate 20. The through holes 20c may improve the stress of the second part 202, increase the flexibility of the second part 202 and thus facilitate the bending extension of the second part 202; accordingly, it is conducive to increasing an extension range and a coverage area of the second parts 202 of the flexible substrate 20 and also helps to improve the adhesion of the probes to the brain of the organism.

In the example of FIG. 4, the flexible substrate 20 includes the first flexible substrate layer 2001 and the second flexible substrate layer 2002 that are arranged in a stacked manner. The through holes 20c avoid the plurality of electrodes 22 and the plurality of leads 23 between the first flexible substrate layer 2001 and the second flexible substrate layer 2002, and run through the first flexible substrate layer 2001 and the second flexible substrate layer 2002. In some embodiments, the through holes 20c are uniformly distributed in the various stages of segments of the second part 202, but other embodiments are possible.

According to some embodiments, the thickness from the 1^{st} to the (N−1)^{th} stage of segments of the plurality of second parts 202 is greater than the thickness of the N^{th} stage of segments of the plurality of second parts 202. In some examples, a difference between the thickness from the 1^{st} to the (N−1)^{th} stage of segments of the plurality of second parts 202 and the thickness of the N^{th} stage of segments of the plurality of second parts 202 may be 5-50 μm, for example, 5 μm, 10 μm, 20 μm, 30 μm, 40 μm, or 50 μm.

In the example of FIG. 4, the second part 202 of the flexible substrate 20 includes two stages of segments, which are the first stage of segment 2021 and the second stage of segments 2022 respectively, that is, N is equal to 2. The thickness of the first stage of segment 2021 is greater than the thickness of the second stage of segments 2022. For example, the second stage of segments 2022 have an additional reinforcement layer 2000 compared with the first stage of segment 2021, and the reinforcement layer 2000 has a thickness d of 5-50 μm.

The presence of the reinforcement layer 2000 may be advantageous. The N^{th} stage of segments of the second part 202 are configured to form the probes that are required to have better flexibility for avoiding brain injuries, and therefore the thickness thereof should not be excessively large. Also, the 1^{st} to the (N−1)^{th} stage of segments of the second part 202 are configured to connect the N^{th} stage of segments and the first part, and by thickening these parts of segments, the strength and the hardness of these parts of segments may be enhanced, the breakage or damage of these parts of segments is avoided, and it is also conducive to preventing the entanglement between the various stages of segments.

In other embodiments, the thickness of the n^{th} stage of segments of the plurality of second parts is greater than the thickness of the (n+1)^{th} stage of segments, where 0<n<N. That is, in the direction from the first end to the second end of the implantable probe apparatus 200, the thicknesses of the plurality of second parts 202 are decreased stage by stage. In this way, it is also possible to avoid the breakage or damage of the 1^{st} to the (N−1)^{th} stage of segments of the second part 202 and to prevent the entanglement between the various stages of segments while the flexibility of the N^{th} stage of segments is ensured and the brain injuries are avoided.

According to some embodiments, the lengths of the segments in the same stage of segments of the second part are not exactly equal. The tail end probes, needing to be implanted, of the respective segments in the same stage of segments have different brain region locations and/or implantation depths, and thus distances between the probes and the probe pad array may also be different. In some embodiments, the lengths of the segments in the same stage of segments may be determined according to the locations of the probe pad array and the implantation regions of the probes, and these lengths need not to be consistent, so that the requirements for the distances between the probe pad array and the implantation regions of the probes can be met. For example, the lengths of the various segments in the second stage of segments 2022 in FIG. 2 are not identical completely, and thus the lengths of the various segments may allow the distances, between the tail end probes corresponding thereto and the probe pad array, to meet implantation requirements.

According to some embodiments, in the second part of the flexible substrate, the numbers of the (n+1)^{th} stage of segments branched from each segment of the n^{th} stage of segments are equal. In this way, the management of the probes can be facilitated. In some examples, each second part is provided with one first stage of segments, 5 branches are branched from the first stage of segments to form a second stage of segments, that is, the number of segments in the second stage of segments is 5. 20 branches are branched from each segment in the second stage of segments to form a third stage of segments, that is, the number of the segments in the third stage of segments branched from each second stage of segments is 20, and the number of the segments in the third stage of segments included in the entire second part is 100. If the third stage of segments are the last stage of segments, tail ends thereof are configured to form probes, and the tail end of the second part is provided with 100 probes. It can thus be seen that the equal numbers of the (n+1)^{th} stage of segments branched from each segment in the n^{th} stage of segments allow a multiplied increase in the numbers of the segments in the various stages of segments, such that the probe management of the last stage of segments is facilitated.

According to some embodiments, the plurality of electrodes in the implantable probe apparatus are deep electrodes for implantation into deep brain regions of an organism. The deep electrodes are used in the deep brain regions and can be configured to detect lesion discharges in the deep brain regions, record intracranial electroencephalograms, etc.

According to some embodiments, the plurality of electrodes in the implantable probe apparatus are cortical electrodes for implantation into the cerebral cortex of an organism. The cortical electrode is applied in a superficial brain region and is an intracranial electrode that is mainly configured to record a cortical potential of the convex surface, the lateral surface or the basilar part of the cerebral hemisphere.

According to some embodiments, the plurality of electrodes in the implantable probe apparatus include both the deep electrodes for implantation into the deep brain regions of an organism and the cortical electrodes for implantation into the cerebral cortex of the organism. For example, in the plurality of second parts of the flexible substrate, some of the electrodes arranged in the tail end segments of the second parts are the deep electrodes, and other electrodes arranged in the tail end segments of the second parts are the cortical electrodes.

As shown in FIGS. 2 and 4, according to some embodiments, the implantable probe apparatus 200 further includes a support substrate 24, and the first part 201 of the flexible substrate 20 is formed on the support substrate 24. In some examples, the support substrate 24 may be a silicon wafer. The first part 201 of the flexible substrate 20 is provided with the probe pad array therein, and the first part 201 is supported by the support substrate 24 to facilitate an operation of connecting the contact pads 21 of the probe pad array to the external circuit, such as a crimping or soldering operation.

As shown in FIG. 2, according to some embodiments, the tail end sections 2020 of the second part 202 are reinforced with a biocompatible material to facilitate the implantation into the brain of the organism. The biocompatible material refers to a material that can be removed, decomposed and dissolved under the influence and action of biological tissues after the implantation into the organism. By way of example but not limitation, the biocompatible material contains silk protein. The tail end sections of the second part are wrapped with a silk protein solution, and after the silk protein solution is solidified, the tail end sections of the second part can be reinforced, and thus the implantation into the brain of the organism is facilitated. After the tail end sections of the second part are implanted into the brain of the organism, the silk protein is dissolved and disappears when encountering a brain tissue fluid, such that the original flexibility of the tail end sections is restored, and the brain injuries can be avoided during the later collection of electrical signals.

Referring to FIG. 5, FIG. 5 is an exploded schematic structural diagram of an electrode apparatus 300 according to some embodiments of the disclosure. As shown in FIG. 5, the electrode apparatus 300 includes a data adapter 30 and an implantable probe apparatus 200 in any one of the embodiments described above.

The data adapter 30 is electrically connected to the plurality of contact pads 21 in the probe pad array and configured to transmit signals to the plurality of contact pads 21 or receive signals from the plurality of contact pads 21. In some examples, the plurality of electrodes of each tail end section of the implantable probe apparatus 200 collect brain tissue signals, transmit the collected signals to the data adapter 30 by means of the contact pads 21, and then transmit the signals to the external circuit by means of the data adapter 30, for example, to a brain signal collection chip. In some examples, the external circuit transmits the signals to the implantable probe apparatus 200 by means of the data adapter 30, and the signals act on the brain tissues by means of the electrodes of the tail end sections of the implantable probe apparatus 200 to output the stimulation signals to the brain tissues.

According to the embodiments of the disclosure, the electrode apparatus 300 includes the implantable probe apparatus 200. The implantable probe apparatus 200 is provided with a large number of probes capable of covering large implantation regions, so that the coverage area of the implantable probe apparatuses 200 can be increased, the number of the implantable probe apparatuses 200 required for electroencephalogram signal detection can be decreased, the number of back-end data adapters 30 required can be decreased, and the skull injuries to the recipient can be reduced.

As shown in FIG. 5, according to some embodiments, the data adapter 30 includes a pad array board 31 and a data interface board 32, and the pad array board 31 is electrically connected to the data interface board 32.

The pad array board 31 includes a plurality of pads 311, and the plurality of pads 311 are electrically connected to the plurality of contact pads 21 in the probe pad array respectively to achieve the electrical connection between the data adapter 30 and the implantable probe apparatus 200. In some embodiments, the pad array board 31 is a PCB.

The data interface board 32 includes a plurality of electrical contacts, and the plurality of electrical contacts are electrically connected to the plurality of pads 311 of the pad array board 31 respectively. In some embodiments, the data interface board 32 functions as a chip interface end that is provided with a specific number (e.g., 4) of chip interfaces 320, a plurality of electrical contacts are provided in each chip interface 320, and a chip (e.g., the brain signal collection chip) may be inserted into the chip interface 320 to achieve communication connection between the chip and the electrode apparatus 300. In some embodiments, the data interface board 32 is a PCB.

As shown in FIG. 5, according to some embodiments, the data adapter 30 further includes a flexible wiring board 33. The flexible wiring board 33 includes a plurality of cables 330, and the plurality of cables 330 electrically connect the corresponding electrical contacts in the plurality of electrical contacts to the respective pads in the plurality of pads 311. In some embodiments, the electrical contacts, the pads 311 and the cables 330 are in one-to-one correspondence with each other, and each cable 330 electrically connects the corresponding electrical contact to the respective pad 311. In some embodiments, the flexible wiring board 33 is a flexible PCB. The flexible wiring board 33 is configured to connect the pad array board 31 to the data interface board 32 in order to achieve a flexible transition between the pad array board 31 and the data interface board 32. In this way, the flexible arrangement of the positions between the implantable probe apparatus 200 and the chip can be facilitated. For example, the chip can be placed vertically relative to the direction of probe implantation of the implantable probe apparatus 200.

In another aspect, the disclosure provides an electronic device. The electronic device includes the electrode apparatus 300 as described above. The electronic device may include, but is not limited to, an implantable neurostimulator, an implantable neurorecorder, an implantable stimulation-recorder, etc.

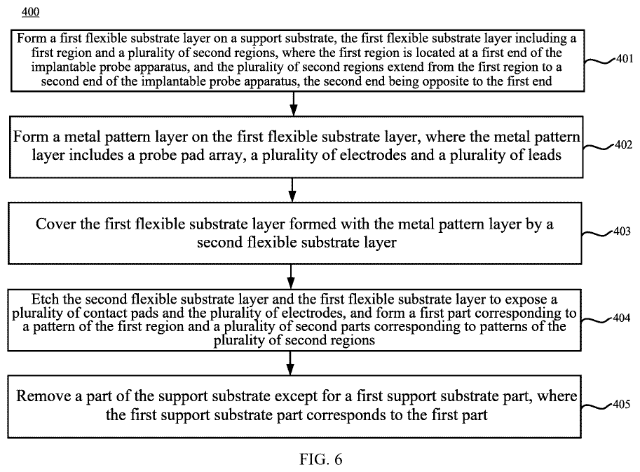

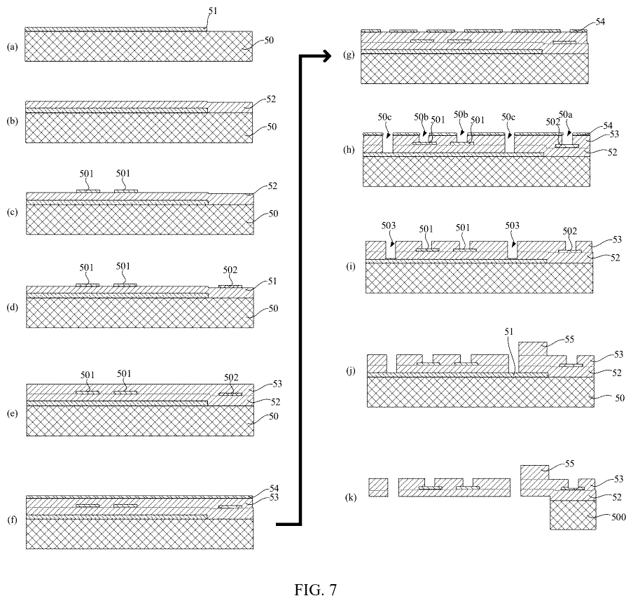

Reference is made to FIGS. 6 and 7. FIG. 6 is a flowchart of a method 400 for preparing an implantable probe apparatus according to some embodiments of the disclosure. FIG. 7 is a schematic diagram of a process for preparing an implantable probe apparatus according to some embodiments of the disclosure.

As shown in FIG. 6, the method 400 includes the following steps.

Step 401: as shown in section (b) of FIG. 7, a first flexible substrate layer 52 is formed on the support substrate 50. The first flexible substrate layer 52 includes a first region and a plurality of second regions, the first region is located at a first end of the implantable probe apparatus, and the plurality of second regions extend from the first region to a second end of the implantable probe apparatus, the second end being opposite to the first end.

Step 402: as shown in sections (c) and (d) of FIG. 7, a metal pattern layer is formed on the first flexible substrate layer 52. The metal pattern layer includes a probe pad array, a plurality of electrodes 501 and a plurality of leads, where the probe pad array includes a plurality of contact pads 502, the plurality of contact pads 502 are formed in the first region, the plurality of electrodes 501 are formed in respective tail end sections of the plurality of second regions away from the first region, and the plurality of leads are formed in the plurality of second regions to electrically connect the corresponding electrodes 501 in the plurality of electrodes 501 to the respective contact pads 502 in the plurality of contact pads 502 respectively.

Step 403: as shown in section (e) of FIG. 7, the first flexible substrate layer 52 formed with the metal pattern layer is covered by a second flexible substrate layer 53. The first flexible substrate layer 52 and the second flexible substrate layer 53 jointly form a flexible substrate layer.

Step 404: as shown in sections (f) to (i) of FIG. 7, the second flexible substrate layer 53 and the first flexible substrate layer 52 are etched to expose the plurality of contact pads 502 and the plurality of electrodes 501, and a first part corresponding to a pattern of the first region and a plurality of second parts corresponding to patterns of the plurality of second regions are formed. In other words, in step 404, the flexible substrate layer is etched to form a pattern of a flexible substrate, where the pattern of the flexible substrate includes a first part and a plurality of second parts, the first part is provided with contact holes 50a for exposing the plurality of contact pads 502, and the plurality of second parts are provided with connecting holes 50b for exposing the plurality of electrodes 501. The plurality of second parts are separated from each other, each second part includes N stages of segments, the N stages of segments are arranged sequentially in a direction from the first end to the second end, the N^{th} stage of segments of the plurality of second parts include tail end sections corresponding to the respective tail end sections of the plurality of second regions, and the tail end sections of the plurality of second parts function as probes for implantation into the brain of an organism, where N represents an integer greater than or equal to 2; also, a plurality of branches are branched from each segment in the n^{th} stage of segments to serve as the (n+1)^{th} stage of segments, and the leads formed in each segment of the (n+1)^{th} stage of segments are subsets of the leads formed in the n^{th} stage of segments, where n represents an integer and 0<n<N.

Step 405: as shown in section (k) of FIG. 7, a part of the support substrate 50 except for a first support substrate part 500 is removed. The first support substrate part 500 corresponds to the first part.

According to the embodiments of the disclosure, the second part of the flexible substrate uses a multi-stage segmentation design, the numbers of segments in the various stages of segments are gradually increased sequentially from the first stage of segment to the N^{th} stage of segments, then the number of segments in the last stage of segments (the N^{th} stage of segments) may be much greater than the number of the segment in the first stage of segment (e.g., amplified exponentially), and the tail end regions of the segments in the last stage of segments are configured as probes. In this way, the implantable probe apparatus is provided with a larger number of probes and thus can cover a larger implantation range, so that the coverage area of a single implantable probe apparatus can be increased. Thus, the number of the implantable probe apparatuses required for detecting electroencephalogram signals can be decreased, the number of rear-end adapter interfaces connected to the implantable probe apparatuses can be decreased, and accordingly the skull injuries to the recipient are reduced.

In addition, in the second part of the flexible substrate, the numbers of segments in the various stages of segments are gradually decreased sequentially from the N^{th} stage of segments to the first stage of segment, so that the grouped management of the probes can be facilitated, and the entanglement between the plurality of leads is prevented.

According to some embodiments, forming a metal pattern layer on the first flexible substrate layer 52 (step 402) includes the following steps.

First, as shown in section (c) of FIG. 7, patterns of the plurality of electrodes 501 and the plurality of leads are prepared in the second regions of the first flexible substrate layer 52 by an etch-patterning process.

Second, as shown in section (d) of FIG. 7, a pattern of the probe pad array is prepared in the first region of the first flexible substrate layer 52 by the etch-patterning process.

According to some embodiments, etching the second flexible substrate layer 53 and the first flexible substrate layer 52 (step 404) further includes: as shown in sections (f) to (i) of FIG. 7, a plurality of through holes 50c running through the second flexible substrate layer 53 and the first flexible substrate layer 52 are etched in the plurality of second parts. In other words, the pattern of the flexible substrate further includes the through holes 50c in the plurality of second parts. The plurality of through holes 50c avoid the plurality of electrodes 501 and the plurality of leads between the first flexible substrate layer 52 and the second flexible substrate layer 53, and run through the first flexible substrate layer 52 and the second flexible substrate layer 53. By arranging the through holes 50c, it is conducive to improving the flexibility of the second parts of the flexible substrate, then increasing a cerebral coverage range of the plurality of second parts of the implantable probe apparatus, and improving the adhesion of the probes of the second parts to the brain of the organism.

According to some embodiments, removing a part of the support substrate 50 except for the first support substrate part 500 (step 405) includes the following steps.

First, as shown in section (a) of FIG. 7, before the first flexible substrate layer 52 is formed, a sacrificial layer 51 is formed on the part of the support substrate 50 except for the first support substrate part 500.

Next, as shown in sections (j) and (k) of FIG. 7, the sacrificial layer 51 is etched off such that the part of the support substrate 50 except for the first support substrate part 500 is separated from the first flexible substrate layer 52, the part of the support substrate 50 except for the first support substrate part 500 is then removed, leaving only the first support substrate part 500 of the support substrate 50 to support the first part of the flexible substrate.

The first part of the flexible substrate is provided with the probe pad array therein, and the first part 201 is supported by the first support substrate part 500 of the support substrate 50, so that an operation of connecting the contact pads 21 of the probe pad array to an external circuit is facilitated. There is no support substrate 50 for supporting under the second parts of the flexible substrate, the second parts may be bent to extend to different regions of the brain, so that the probes of the tail end segments of the second parts can be implanted into the different regions of the brain.

According to some embodiments, the method 400 for preparing the implantable probe apparatus further includes the following steps. As shown in section (j) of FIG. 7, before the part of the support substrate 50 except for the first support substrate part 500 is removed, a flexible substrate reinforcement layer 55 is formed on the 1^{st} to (N−1)^{th} stage of segments of the plurality of second parts. In some examples, the reinforcement layer 55 has a thickness d of 5-50 μm.

The N^{th} stage of segments of the second part are configured to form the probes that are required to have better flexibility for avoiding brain injuries, and therefore the thickness thereof should not be excessively large. Also, the 1^{st} to the (N-1)^{th} stage of segments of the second part are configured to connect the N^{th} stage of segments and the first part, and by thickening these parts of segments, the strength and the hardness of these parts of segments are enhanced, the breakage or damage of these parts of segments is avoided, and it is also conducive to preventing the entanglement between the various stages of segments.

A specific example of the method 400 for preparing the implantable probe apparatus will be described in detail below in conjunction with FIG. 7.

As shown in section (a) of FIG. 7, a patterned sacrificial layer 51 is deposited on the support substrate 50. This step may include the following procedure:
1) apply a photoresist and pattern the photoresist to form a sacrificial layer arrangement region;
2) deposit chromium (Cr) and nickel (Ni) in the sacrificial layer arrangement region by using a metal evaporation method to form the sacrificial layer, where the thicknesses of the chromium (Cr) and the nickel (Ni) are Cr=25-75 Å and Ni=250-750 Å respectively; and angstrom (Å) represents a length unit, and 1 angstrom=0.1 nanometer.
3) use acetone to peel off the photoresist, and remove the metal layer on the photoresist together therewith, leaving only the sacrificial layer in the sacrificial layer arrangement region after peeling off.

As shown in section (b) of FIG. 7, the patterned sacrificial layer is spin-coated with the first flexible substrate layer 52, and the first flexible substrate layer 52 is cured by means of a stepped temperature increase of a vacuum oven. For example, the first flexible substrate layer 52 is made of polyimide (PI) having a thickness of 1-10 μm and a maximum curing temperature of 380° C.

As shown in section (c) of FIG. 7, the electrodes 501 and the leads are prepared on the first flexible substrate layer 52. This step may include the following procedure:
1) apply a photoresist and pattern the photoresist to form an electrode and lead arrangement region, where the arrangement region is located on the second regions of the first flexible substrate layer 52;
2) deposit chromium (Cr) and gold (Au) on the electrode and lead arrangement region by means of a metal evaporation method to form the electrodes and the leads; where the thicknesses of the chromium (Cr) and the gold (Au) are Cr=5-50 nm and Au=50-500 nm respectively; and
3) use acetone to peel off the photoresist, and remove the metal layer on the photoresist together therewith, leaving only the electrodes and the leads in the arrangement region after peeling off.

As shown in section (d) of FIG. 7, a contact bonding spot 502 is prepared on the first flexible substrate layer 52, a preparation process thereof is the same as the preparation process of the electrodes 501 and the leads, while differences therebetween lie in that the arrangement region of the contact bonding spot 502 is located in the first region of the first flexible substrate layer 52, and a metal evaporation layer thereof includes three layers of chromium (Cr), nickel (Ni) and gold (Au) that have thicknesses of Cr=5-50 nm, Ni=100-1500 nm and Au=50-500 nm respectively; and

as shown in section (e) of FIG. 7, the second flexible substrate layer 53 (i.e., an encapsulation layer) is prepared on the electrodes 501, the leads and the contact bonding spot 502, and the second flexible substrate layer 53 is cured by means of a stepped temperature increase of the vacuum oven. For example, the second flexible substrate layer 53 is made of polyimide (PI) having a thickness of 2-20 μm and a maximum curing temperature of 380° C. In this case, the electrodes, the leads and the contact bonding spot are all encapsulated in the flexible substrate layer.

As shown in section (f) of FIG. 7, an aluminum hardmask layer 54 is formed on the second flexible substrate layer 53 by using a sputtering technology, and the thickness thereof is 50-200 nm.

As shown in section (g) of FIG. 7, the aluminum hardmask layer 54 is patterned. This step may include the following procedure:

1) apply a photoresist to an aluminum layer, and pattern the photoresist to form a region to be etched;

2) etch the aluminum layer in the region to be etched by using an aluminum etching solution, leaving part of the aluminum layer covered by the photoresist to be not etched; and
3) remove the residual photoresist, leaving a patterned aluminum layer to serve as a mask layer for etching the first flexible substrate layer and the second flexible substrate layer.

As shown in section (h) of FIG. 7, the first flexible substrate layer 52 and the second flexible substrate layer 53 are etched by using the patterned aluminum hardmask layer 54 as the mask. This step may include the following procedure:

a PI layer (the first flexible substrate layer 52 and the second flexible substrate layer 53) in the region to be etched (a region that is not covered by the aluminum hardmask layer 54) is etched by using a deep silicon etching technique, where a single laterally-etched side of the PI layer etched is ±0.5 μm; and after the PI layer is etched, the patterns of the first part and the second parts, the connecting holes 50b for exposing the electrodes 501, and the contact holes 50a for exposing the contact bonding spots 502 may be formed. In addition, the through holes 50c running through the PI layer may be formed.

The patterned aluminum hardmask layer 54 is removed by using the aluminum etching solution, and a structure after the aluminum hardmask layer 54 is removed is as shown in section (i) of FIG. 7; and

as shown in section (j) of FIG. 7, by using spin coating and photolithographic patterning techniques again, the reinforcement layer 55 is formed on the 1^{st} to (N−1)^{th} stage of segments of each second part of the flexible substrate, and the reinforcement layer 55 is made from polyimide (PI) and has a thickness of 5-50 μm. For the procedure of the photolithographic patterning technique for the reinforcement layer, reference may be made to the photoetching process of the flexible substrate layer, which will not be described in detail herein.

As shown in section (j) of FIG. 7, the sacrificial layer 51 is etched by using an etching solution, the support substrate part corresponding to the sacrificial layer 51 is removed, only the first support substrate part 500 is left to support the first part of the flexible substrate, and the structure formed after the support substrate part corresponding to the sacrificial layer 51 is removed is as shown in section (k) of FIG. 7.

It should be noted that the above preparation steps are merely illustrative for the preparation method 400, and the preparation method 400 is not limited to the above-described embodiments and may specifically be adjusted according to actual process requirements.

The implantable probe apparatus according to the embodiments of the disclosure and the preparation method therefor are on the basis of the same inventive concept, and therefore the preparation method according to the embodiments of the disclosure also has the same or similar beneficial effects as the implantable probe apparatus described above, which will not be described in detail herein.

In this description, the orientations or positional relationships or dimensions denoted by the terms, such as “center”, “longitudinal”, “transverse”, “length”, “width”, “thickness”, “upper”, “lower”, “front”, “rear”, “left”, “right”, “vertical”, “horizontal”, “top”, “bottom”, “inner”, “outer”, “clockwise”, “counterclockwise”, “axial”, “radial” and “circumferential”, are the orientations or positional relationships or dimensions shown on the basis of the accompanying drawings, and these terms are used merely for ease of description, rather than indicating or implying that the apparatus or element referred to must have particular orientations and be constructed and operated in the particular orientations, and therefore should not be construed as limiting the scope of protection of the disclosure.

In addition, the terms such as “first”, “second” and “third” are merely for descriptive purposes and should not be construed as indicating or implying relative importance or implicitly indicating the number of technical features indicated. Thus, the features defined with “first”, “second” and “third” may explicitly or implicitly include one or more features. In the description of the disclosure, the term “a plurality of” means two or more, unless otherwise explicitly and specifically defined.

In the disclosure, unless expressly stated or defined otherwise, the terms such as “mounting”, “connection”, “connected” and “fixing” should be interpreted broadly, for example, they may be a fixed connection, a detachable connection, or an integrated connection; may be a mechanical connection, or an electrical connection, or communication; and may be a direct connection or an indirect connection by means of an intermediate medium, or may be internal communication between two elements or interaction between the two elements. For those of ordinary skill in the art, the specific meanings of the above terms in the disclosure can be understood according to specific circumstances.

In the disclosure, unless expressly stated or defined otherwise, the expression of the first feature being “above” or “below” the second feature may include the case that the first feature is in direct contact with the second feature, or the case that the first feature and the second feature are not in direct contact but are contacted via another feature therebetween. Furthermore, the first feature being “over”, “above” or “on” the second feature includes the case where the first feature is directly or obliquely above the second feature, or merely indicates that the first feature is at a higher level than the second feature. The first feature being “below”, “under” or “beneath” the second feature includes the case where the first feature is directly or obliquely below the second feature, or merely indicates that the first feature is at a lower level than the second feature.

This description provides many different implementations or examples that can be used to implement the disclosure. It should be understood that these different implementations or examples are purely illustrative and are not intended to limit the scope of protection of the disclosure in any way. On the basis of the disclosure of the description of the disclosure, those skilled in the art will be able to conceive of various changes or substitutions. All these changes or substitutions shall fall within the scope of protection of the disclosure. Accordingly, the scope of protection of the disclosure shall be subject to the scope of protection of the claims.

1. An implantable probe apparatus, comprising:
a flexible substrate, which comprises a first part and a plurality of second parts separated from each other, wherein the first part is located at a first end of the implantable probe apparatus, and the plurality of second parts extend from the first part to a second end of the implantable probe apparatus, the second end being opposite to the first end;
a probe pad array, which comprises a plurality of contact pads that are formed in the first part;
a plurality of electrodes, which are formed in tail end sections of the plurality of second parts away from the first part, the tail end sections serving as probes to be implanted into the brain of an organism; and
a plurality of leads, which are formed in the plurality of second parts to electrically connect respective electrodes in the plurality of electrodes to corresponding contact pads in the plurality of contact pads respectively;
wherein each second part in the plurality of second parts comprises N stages of segments, the N stages of segments are arranged sequentially in a direction from the first end to the second end, and the N^{th} stage of segments of the plurality of second parts comprise the tail end sections of the plurality of second parts, where N represents an integer greater than or equal to 2; and
wherein a plurality of branches are branched from each segment in the n^{th} stage of segments to serve as the (n+1)^{th} stage of segments, and the leads formed in each segment of the (n+1)^{th} stage of segments are subsets of the leads formed in the n^{th} stage of segments, where n represents an integer and 0<n<N.

2. The implantable probe apparatus according to claim 1, wherein the plurality of second parts comprise a plurality of through holes running through the flexible substrate.

3. The implantable probe apparatus according to claim 1, wherein the thickness from the 1^{st} to the (Nμ1)^{th} stage of segments of the plurality of second parts is greater than the thickness of the N^{th} stage of segments of the plurality of second parts; or
the thickness of the n^{th} stage of segments of the plurality of second parts is greater than the thickness of the (n+1)^{th} stage of segments of the plurality of second parts.

4. The implantable probe apparatus according to claim 1, wherein the lengths of the segments in the same stage of segments are not exactly equal.

5. The implantable probe apparatus according to claim 1, wherein the numbers of the (n+1)^{th} stage of segments branched from each segment of the n^{th} stage of segments are equal.

6. The implantable probe apparatus according to claim 1, wherein the plurality of electrodes are deep electrodes for implantation into deep brain regions of an organism.

7. The implantable probe apparatus according to claim 1, wherein the plurality of electrodes are cortical electrodes for implantation into the cerebral cortex of an organism.

8. The implantable probe apparatus according to claim 1, further comprising: a support substrate on which the first part of the flexible substrate is formed.

9. The implantable probe apparatus according to claim 1, wherein the tail end sections are reinforced with a biocompatible material to facilitate the implantation into the brain of the organism.

10. The implantable probe apparatus according to claim 9, wherein the biocompatible material contains silk protein.

11. An electrode apparatus, comprising:
an implantable probe, comprising:
a flexible substrate, which comprises a first part and a plurality of second parts separated from each other, wherein the first part is located at a first end of the implantable probe apparatus, and the plurality of second parts extend from the first part to a second end of the implantable probe apparatus, the second end being opposite to the first end;
a probe pad array, which comprises a plurality of contact pads that are formed in the first part;
a plurality of electrodes, which are formed in tail end sections of the plurality of second parts away from the first part, the tail end sections serving as probes to be implanted into the brain of an organism; and
a plurality of leads, which are formed in the plurality of second parts to electrically connect respective electrodes in the plurality of electrodes to corresponding contact pads in the plurality of contact pads respectively;
wherein each second part in the plurality of second parts comprises N stages of segments, the N stages of segments are arranged sequentially in a direction from the first end to the second end, and the N^{th} stage of segments of the plurality of second parts comprise the tail end sections of the plurality of second parts, where N represents an integer greater than or equal to 2; and
wherein a plurality of branches are branched from each segment in the n^{th} stage of segments to serve as the (n+1)^{th} stage of segments, and the leads formed in each segment of the (n+1)^{th} stage of segments are subsets of the leads formed in the n^{th} stage of segments, where n represents an integer and 0<n<N; and
a data adapter, which is electrically connected to the plurality of contact pads in the probe pad array and configured to transmit signals to the plurality of contact pads or receive signals from the plurality of contact pads.

12. The electrode apparatus according to claim 11, wherein the plurality of second parts comprise a plurality of through holes running through the flexible substrate.

13. A method for preparing an implantable probe apparatus, the method comprising:
forming a first flexible substrate layer on a support substrate, the first flexible substrate layer comprising a first region and a plurality of second regions, wherein the first region is located at a first end of the implantable probe apparatus, and the plurality of second regions extend from the first region to a second end of the implantable probe apparatus, the second end being opposite to the first end;
forming a metal pattern layer on the first flexible substrate layer, the metal pattern layer comprising a probe pad array, a plurality of electrodes and a plurality of leads, wherein the probe pad array comprises a plurality of contact pads, the plurality of contact pads are formed in the first region, the plurality of electrodes are formed in respective tail end sections of the plurality of second regions away from the first region, and the plurality of leads are formed in the plurality of second regions to electrically connect the corresponding electrodes in the plurality of electrodes to the respective contact pads in the plurality of contact pads respectively;
covering the first flexible substrate layer formed with the metal pattern layer by a second flexible substrate layer;
etching the second flexible substrate layer and the first flexible substrate layer to expose the plurality of contact pads and the plurality of electrodes, and forming a first part corresponding to a pattern of the first region and a plurality of second parts corresponding to patterns of the plurality of second regions, wherein the plurality of second parts are separated from each other, each second part comprises N stages of segments, the N stages of segments are arranged sequentially in a direction from the first end to the second end, the N^{th} stage of segments of the plurality of second parts comprise tail end sections corresponding to the respective tail end sections of the plurality of second regions, and the tail end sections of the plurality of second parts function as probes for implantation into the brain of an organism, where N represents an integer greater than or equal to 2; and wherein a plurality of branches are branched from each segment in the n^{th} stage of segments to serve as the (n+1)^{th} stage of segments, and the leads formed in each segment of the (n+1)^{th} stage of segments are subsets of the leads formed in the n^{th} stage of segments, where n represents an integer and 0<n<N; and
removing a part of the support substrate except for a first support substrate part, the first support substrate part corresponding to the first part.

14. The method according to claim 13, wherein etching the second flexible substrate layer and the first flexible substrate layer comprises:
etching the plurality of second parts to form a plurality of through holes running through the second flexible substrate layer and the first flexible substrate layer.

15. The method according to claim 13, further comprising:
before the part of the support substrate except for the first support substrate part is removed, forming a flexible substrate reinforcement layer on the 1^{st} to (N−1)^{th} stage of segments of the plurality of second parts.

16. The electrode apparatus according to claim 11, wherein the thickness from the 1^{st} to the (N−1)^{th} stage of segments of the plurality of second parts is greater than the thickness of the N^{th} stage of segments of the plurality of second parts; or
the thickness of the n^{th} stage of segments of the plurality of second parts is greater than the thickness of the (n+1)^{th} stage of segments of the plurality of second parts.

17. The electrode apparatus according to claim 11, wherein the lengths of the segments in the same stage of segments are not exactly equal.

18. The electrode apparatus according to claim 11, wherein the numbers of the (n+1)^{th} stage of segments branched from each segment of the n^{th} stage of segments are equal.

19. The electrode apparatus according to claim 11, wherein the plurality of electrodes are deep electrodes for implantation into deep brain regions of an organism.

20. The electrode apparatus according to claim 11, wherein the plurality of electrodes are cortical electrodes for implantation into the cerebral cortex of an organism.
